(19) United States
(12) Patent Application Publication
(10) Pub. No.: US 2016/0120894 A1
(43) Pub. Date: May 5, 2016

(54) USE OF ADENOSINE ASPARTATE IN THE DIFFERENTIAL ACTIVATION OF MACROPHAGES IN INFLAMMATORY-FIBROGENIC PROCESSES AND ITS REVERSAL

(71) Applicant: UNIVERSIDAD NACIONAL AUTÓNOMA DE MÉXICO, Distrito Federal (MX)

(72) Inventors: Victoria Eugenia CHAGOYA Y HAZAS, Distrito Federal (MX); Rebeca PEREZ CABEZA DE VACA, Distrito Federal (MX); Mariana DOMINGUEZ LOPEZ, Distrito Federal (MX); Francisco HERNANDEZ LUIS, Distrito Federal (MX)

(21) Appl. No.: 14/893,315

(22) PCT Filed: May 20, 2014
(86) PCT No.: PCT/MX2014/000073

§ 371 Date: Nov. 23, 2015

(30) Foreign Application Priority Data
May 22, 2013 (MX) MX/A/2013/005735

(51) Int. Cl. A61K 31/7076

(52) U.S. Cl. CPC A61K 31/7076

(57) ABSTRACT

The present invention relates to the use of adenosine aspartate for differential activation of macrophages in inflammatory-fibrogenic processes and its reversal.

### FIELD

The present invention lies in the chemical and pharmaceutical field. Specifically, the present invention relates to the use of adenosine aspartate, referred to herein as IFC-305, in the differential activation of macrophages in inflammatory-fibrogenic processes and its reversal.

### BACKGROUND

The immune response is intended to maintain tissue homeostasis after injury, generating a self-regulated inflammatory response. When the injury becomes chronic, inflammatory response also activates chronically, creating a change in the structure and function of the organ as in cirrhosis and multiple diseases associated with fibrosis.

The liver is an organ with a predominantly innate immunity (Gao B, Jeong W I, Tian Z. Hepatology: 47:729, 2008). Recently they have been linked directly to Kupffer cells and changes in their phenotype and activation to the resolution of hepatic fibrosis one of the major liver diseases. The search for new therapeutic options focuses its efforts to find molecules that modulate the phenotype of activation of Kupffer cells. (Ramachandran P, Iredale J P. J Hepatol: 56:1417, 2012).

Thus, it was found that all cells of the immune response have specific receptors for adenosine, considering a relevant therapeutic target (Haskó, G et al. Nat Rev Drug Discov; 7:759, 2008. Haskó, G, Cronstein B N. Trends Immunol: 25:33, 2004).

Accordingly, the US patent application no. 2012/0039946 A1 discloses a composition that enhances innate immunity of a subject, said composition comprising an immunostimulant and an ingredient derived from degradation of a bacterium, the composition is called ligand MRE, which plays an important role in the switch of a macrophage activity M1 to a macrophage activity M2. However, the disadvantage is as indicated in paragraph [0085], where described the obtaining of the origin strain, later to make an enzymatic digestion and obtain the product, this document is directed to an exacerbation of the phenotype M1 (0163, 0207, 0296) contributing, inter alia, in phagocytic activities.

Likewise, the US patent application no. 2012/0207790 A1 discloses an immunosuppressive agent comprising cells that proliferated by culturing pelleted cells, obtained in turn from centrifugation of adipose tissue to increase the macrophages M2 by transforming M1 to M2 and their use for scleroderma treatment, nephritis, systemic lupus erythematosus or inflammatory lung disorder. However, the use of cell therapy in many countries has two drawbacks: the first is the lack of clinical experience for its application in humans, and on the other hand, the design of the infrastructure for the production and maintenance of stem cells.

Application WO/2012/056251 describes a composition comprising a therapeutically effective amount of a compound which is capable of increasing the production of interleukin-10 (IL-10), said document has no substantial data to quantify the amount of macrophages even though they proposed its participation in the observed mechanism. Its solid part is the detection of IL-10, which performs only in lung tissue, so it would not be reflecting a systemic event.

The US patent application no. 2012/0258135 describes a method of making an immunogenic composition for intradermal or subcutaneous administration. According to the description of the invention, the generation of an immune reaction may include the transformation or activation of a macrophage M1 to Macrophage M2. However, throughout the description can be determined that the downside is itself the immunogenic composition from bacterial or viral determinants, as these determinants are organ-specific, and the preparation and administration should be specific for each organ as required and the initial way generate an infection that requires the co-administration of an anti-inflammatory to slow it down. This activation of the immune response to infection is not controlled and when it is exacerbated, could generate irreversible tissue damage; Side effects are greater as with other immunogenic vaccines, there is fever and irritation at the site of administration.

### SUMMARY

Therefore, an object of the present invention relates to the use of a drug compound which is adenosine aspartate, referred to herein as IFC-305, in the differential activation of macrophages in inflammatory-fibrogenic processes and its reversal.

Also an object of the present invention is to provide a pharmacological compound, adenosine aspartate, which has inducing activity of a phenotype macrophage M2.

A further object of the present invention is to provide pharmaceutical compositions containing adenosine aspartate in therapeutically effective amounts for treating inflammatory-fibrogenic disorders.

Furthermore, an object of the present invention is to provide a pharmaceutical composition containing adenosine aspartate, wherein said composition may be administered in a wide range, without side effects, no toxicity and no teratogenicity nor carcinogenicity.

### BRIEF DESCRIPTION OF THE DRAWINGS

FIGS. 1A to 1C refer to the obtained results for serum cytokine evaluated by Luminex® at 4, 6 and 8 weeks of administration of CCl₄ and CCl₄+IFC. Concentration (pg/mL)±standard deviation is shown.

FIGS. 2A and 2B refer to the results obtained for cytokines in liver homogenate evaluated by Luminex® at 8 weeks of administration of CCl₄ and CCl₄+IFC-305. Concentration (pg/mL)±standard deviation is shown.

FIGS. 3A to 3C refer to the obtained results for serum cytokines evaluated by Luminex® in the different experimental groups of cirrhotic animals (Ci) treated with IFC-305 and saline for five and ten weeks (Ci SS 5s, Ci IFC 5s, Ci SS 10s and Ci IFC 10s post-treatment with CCl₄). Concentration (pg/mL)±standard deviation is shown.

FIGS. 4A to 4D refer to the obtained results for cytokines in liver homogenate evaluated by Luminex® in the different experimental groups of cirrhotic animals (Ci) treated with IFC-305 and saline for five and ten weeks (Ci SS 5s, Ci IFC 5s, Ci SS 10s, and Ci IFC 10s). Concentration (pg/mL)±standard deviation is shown.

FIG. 5 refers to the kinetic of Kupffer cells (CD163+/CD11b+) in the liver, assessed by flow cytometry at 4, 6, 8 and 10 weeks, administration with CCl₄, IFC-305 and CCl₄+IFC-305 to same time. % Kupffer cells±standard error.

FIGS. 6A and 6B relate to the obtained results regarding the distribution of macrophages. FIG. 6A shows total macrophages (HIS36+/CD11b+) in the liver and FIG. 6B shows inflammatory phenotype Kupffer cells (CD163+/CD11b/c+(M1)) evaluated by flow cytometry for 10 weeks of treatment with CCl₄ (Ci) and five and ten weeks with saline (SS) and IFC-305 (IFC) respectively. Percentage (%) of Kupffer cells with respect to control±standard error.

FIG. 7 refers to Western blot Arginase I. Expression of total protein in liver homogenate in the different experimental groups treated with IFC-305 and saline for five and ten weeks.

### DETAILED DESCRIPTION

The present invention relates to the use of adenosine aspartate, referred to herein as IFC-305, in the differential activation of macrophages in inflammatory-fibrogenic processes and its reversal.

According to the present invention, the adenosine aspartate is a pharmochemical compound having by itself the capacity to induce PPAR gamma. The authors of the present invention have found that adenosine aspartate is inducing of the phenotype macrophage M2 and consequently represents a pharmacological option for inflammatory-fibrogenic diseases.

In the prior art it is known that the reaction M1 is induced with pathogens and switching to M2 by to the addition of IL-4 and IL-13; one speaks then of the interconversion M1→M2 through endogenous adenosine as an independent alternative mechanism of IL-4/1L-13. However, the present invention discloses the reduction of the macrophages M1 and achieved the conversion to M2 by exogenous adenosine aspartate, administered in high concentrations (relative to cells), without involve side reactions and/or toxicity to the organism; actually the adenosine aspartate has a longer life than adenosine, and is effective at a lower dose (in relation to a administered dose to human beings) compared to said compound. According to the present invention, the immunomodulatory effect of adenosine, base molecule of the compound, is primarily mediated by the adenosine receptors (A1, A2a, A2b and A3), derivative salts of adenosine can then interact with receptors thanks to metabolism of the molecule or to the cell energy balance to modulate differential activation of macrophages and immune response.

Also, the inventors of the present invention found that such adenosine salt is capable of reacting in any organ, since virtually all human cells possess adenosine receptors or transporters, so that the administration may be by any via, without represent an immunological hyper-reactivation risk.

According to the present invention, the adenosine aspartate may be contained in a pharmaceutical composition, which must contain a therapeutically effective amount of the adenosine aspartate in combination with one or more pharmaceutically acceptable excipients.

As previously described, due to that the adenosine aspartate has a longer life than endogenous adenosine, is effective at a lower dose.

According to the present invention, a series of experimental pre-clinical testing were performed to demonstrate the hepatoprotective effect and the tissue regeneration of the adenosine aspartate (IFC-305) in the induction of cirrhosis with CCl₄ and during the reversal.

Also, the participation of Kupffer cells and the immune response in the cirrogenic process induced with CCl₄ was demonstrated.

### Example 1

Cirrhosis is a disease chronic degenerative characterized by diffuse fibrosis and regenerative nodules distorting tissue architecture. The diffuse fibrosis is supported by a chronic inflammatory process. During this process, chemokines and cytokines that favor this process are generated. In a model of prevention of cirrhosis by treatment with adenosine aspartate, knowing having an antifibrogenic effect, would allow knowing the effect of this drug during the establishment of cirrhosis for 4, 6 and 8 weeks and its effect on response activation immune and inflammation.

In this study, four groups of six male rats of 100 g to 110 g were formed; the rats were treated simultaneously under the same doses with CCl₄ (25 μl of carbon tetrachloride in vegetable oil) and IFC-305 (50 mg/Kg). All treatments were given three times a week for 4, 6 and 8 weeks; at the moment of sacrifice, of serum and liver were taken.

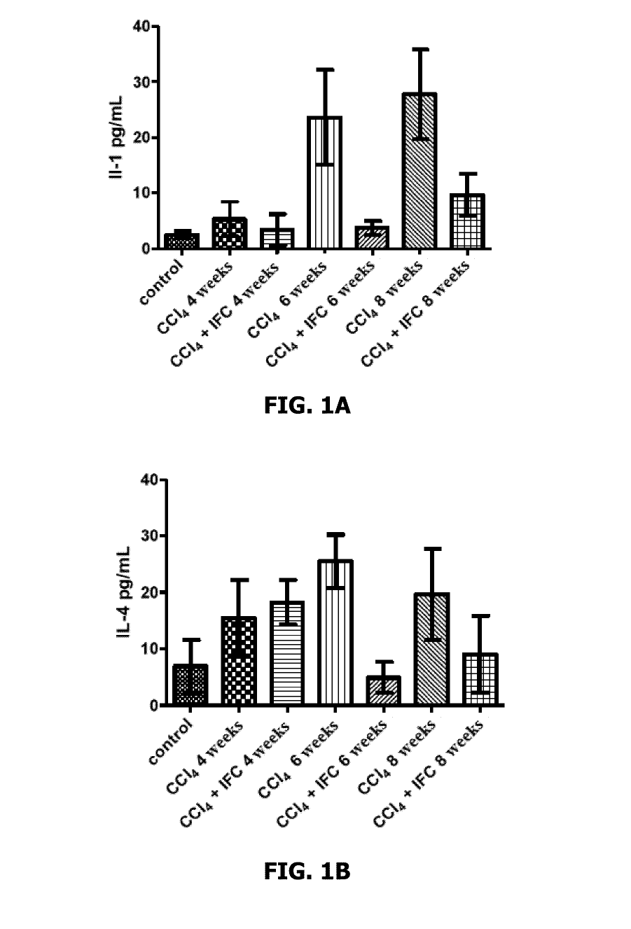

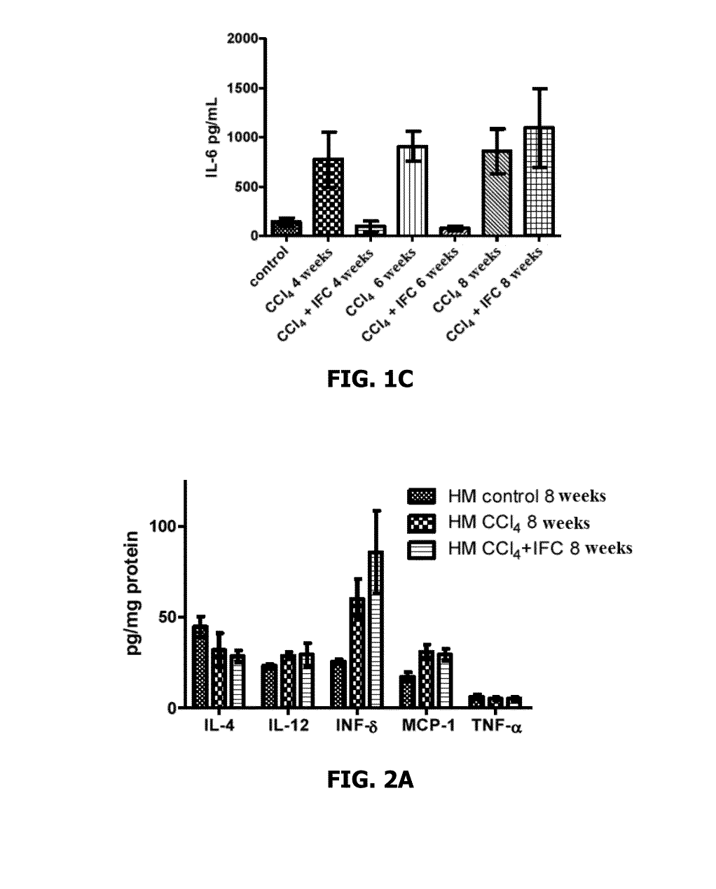

In FIGS. 1A to 1C, is clearly seen the increasing of the pro-inflammatory cytokines (IL-1β-FIG. 1A, IL-4-FIG. 1B and IL-6-FIG. 1C) at 4, 6 and 8 weeks of induction cirrhosis with CCl₄ and the decreasing thereof, in the presence of adenosine aspartate; except IL-6 at 8 weeks of treatment, showing the anti-inflammatory effect of the compound.

Results in serum indicate decreased of the pro-inflammatories cytokines IL-4 showing a clearly antiinflammatory effect. In liver homogenate the same effect at 8 weeks is observed. The dual effect of IL-6 is observed by a decrease at 6 weeks and an increase in serum and liver at 8 weeks, indicating changes in the pattern of secretion of proinflammatory and profibrogenic molecules by administering the compound, protecting the liver of an inflammatory and fibrogenic process.

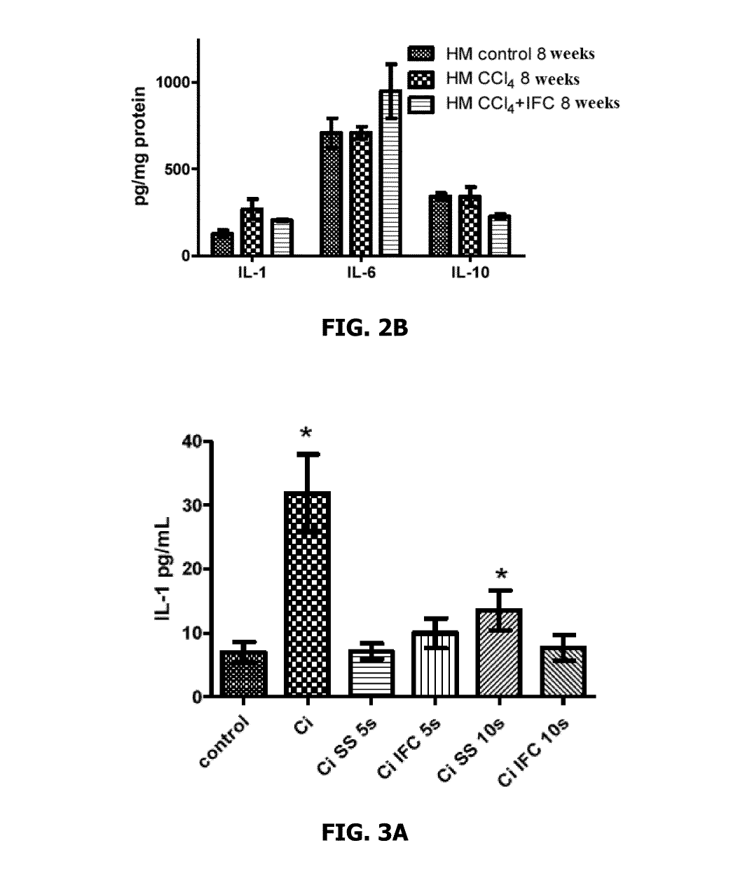

FIGS. 2A and 2B show the results of liver homogenate obtained. Observed that treatment with CCl₄ increases IL-1β at 8 weeks, not with the IL-4 and IL-10, the treatment with adenosine aspartate (referred IFC-305) decreases the three cytokines as in serum. Moreover, IL-6, IL-12, INF-γ and MCP-1 increase relative to the control, because the IFC-305 treatment contributes to the activation and recruitment of monocytes, and with the macrophages differentiation.

### Example 2

This example allows demonstrate the effect of the administration of the IFC-305 in the immune response, present in an established cirrhosis when no longer participates in the inflammatory process and which has been demonstrated the reversion of the induced cirrhosis with CCl₄, normalization of liver function and recovery of liver regeneration.

100 g male Wistar rats were used, with free access to food and water. Cirrhosis was induced with 25 μl of CCl₄ in vegetable oil three times a week for ten weeks. Once cirrhosis is established, the animals were divided into five groups: group I, treatment with CCl₄ is suspended, control animals of cirrhosis are sacrificed, groups II and III is allowed to proceed cirrhosis and received three intraperitoneal injections of saline per week for 5 to 10 weeks, groups IV and V received three intraperitoneal injections of the compound at a dose of 50 mg/kg for 5 to 10 weeks; at the moment of sacrifice, serum and liver samples taken.

By quantifying inflammatory cytokines in serum was observed that the IL-4 and IL-6 increased at 5 weeks and at 10 weeks increases IL-1 β, animals treated with IFC-305 are kept in similar values to controls or less. In liver homogenate, the cirrhosis high values are also reduced by the treatment. Interestingly, treatment with the compound increases levels of anti-inflammatory IL-10.

By quantifying total tissue macrophages (HIS36+), they decreased markedly during cirrhosis and as they progresses, they increase, being more evident the increased presence of the drug.

In evaluating Kupffer cells with inflammatory phenotype by flow cytometry with the marker (CD163+/CD11b/c+) decrease was observed during the course of cirrhosis that is increased in the presence of the compound. These observations suggest that the detected macrophages, that increasing in the presence of the drug are not inflammatory but actually refer to macrophages with alternative activation (M2). To test this possibility, the amount of arginase was evaluated, which was found increased accordingly to what happens in the Macrophage M2.

Thus, we can conclude that during the reversal of cirrhosis by the IFC-305, the immune response is modified promoting the tissue recovery by reducing the fibrogenesis and the oxidative stress. By modifying the activation state of M1 to M2 in Kupffer cells.

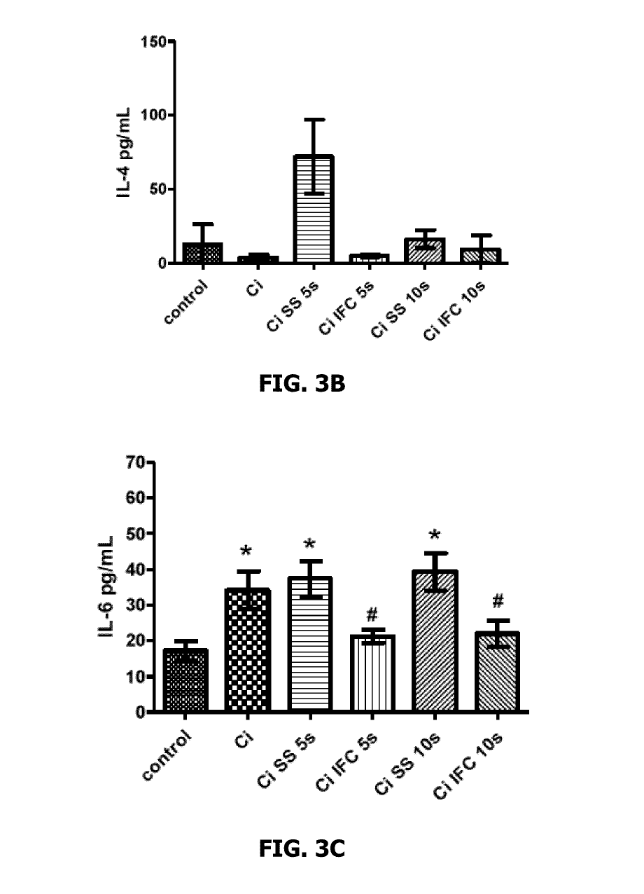

FIGS. 3A to 3C relate to the evaluation of inflammatory cytokines (IL-1β, IL-4, IL-10) in serum during the reversal of cirrhosis, finding increased IL-4 and IL-6 at 5 weeks of cirrhosis progression and of IL-1 β at 10 weeks. In the presence of IFC-305 they were maintained in an equal range to the control or lower.

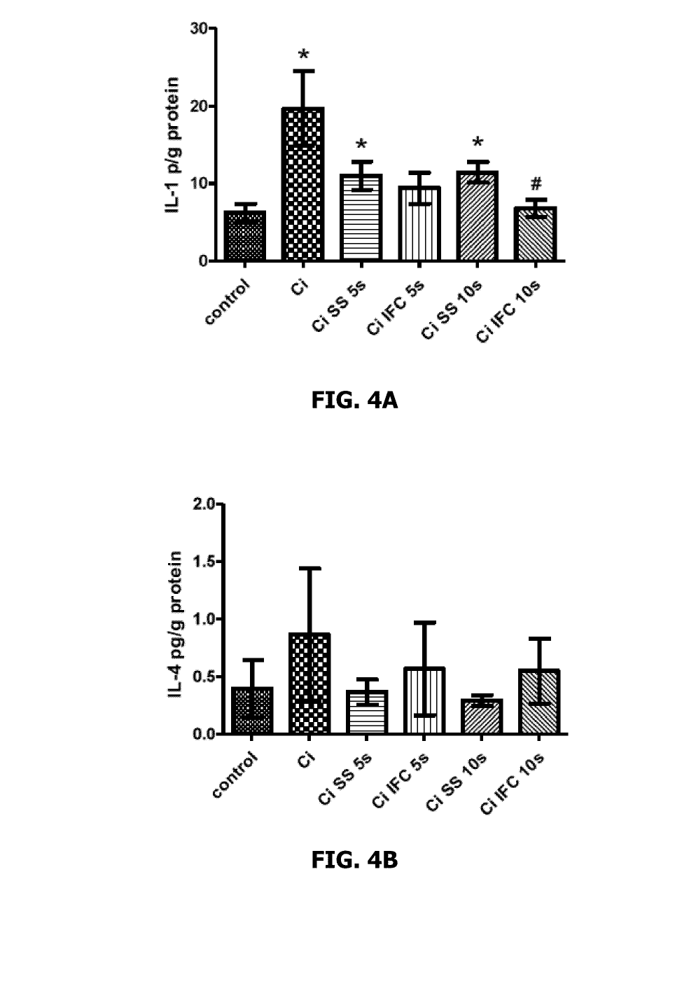

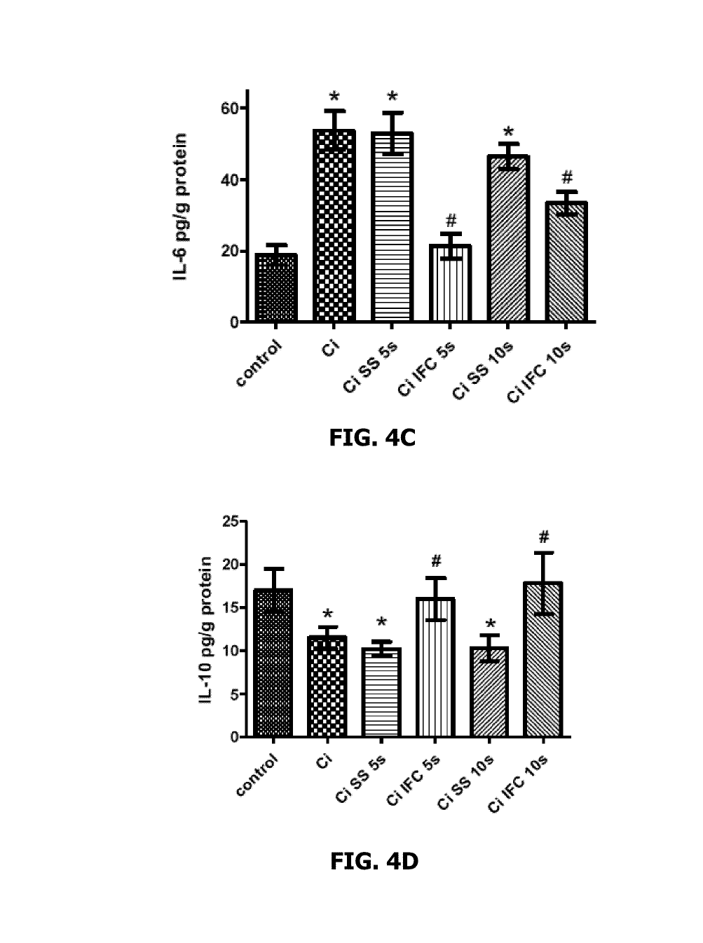

In FIGS. 4A to 4C it is observed that the inflammatory cytokines IL-1β and IL-4 decreased compared to cirrhotic control in the absence or presence of the drug. The IL-6 cytokine activity both pro- and anti-inflammatory is regulated with IFC-305 treatment at five and ten weeks, indicating that in these cases has anti-inflammatory activity. The anti-inflammatory cytokine IL-10 decreases during the progression of cirrhosis and increased by the presence of the drug.

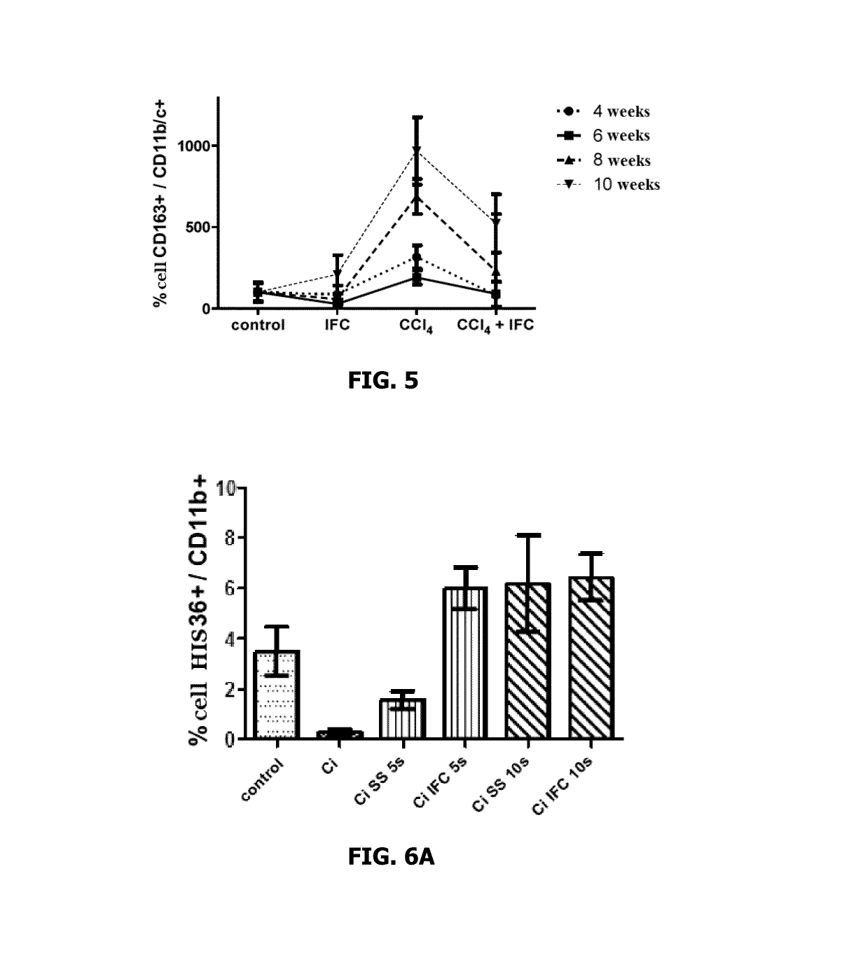

FIG. 5 refers to a graphic showing the total number of Kupffer cells quantified with CD163+/CD11b/c+ for 4, 6, 8 and 10 weeks of treatment with CCl₄, with an increase during the development of cirrhosis that decreases with the treatment of the compound IFC-305.

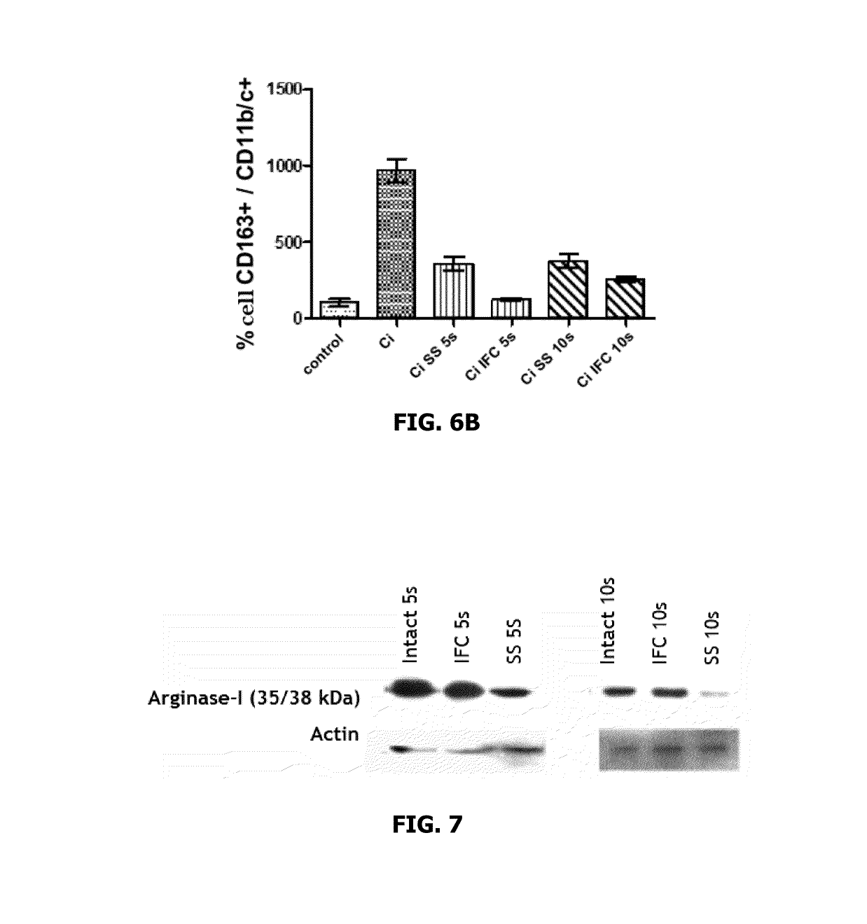

FIGS. 6A and 6B refer to the results of liver homogenate with regard to the distribution of macrophages. FIG. 6A refers to the total macrophages using the marker HIS36+/CD11b+, where a significant decrease in cirrhosis and increases during progression of cirrhosis was observed and with the treatment of the IFC-305. FIG. 6B relates to the quantification of Kupffer cells with inflammatory phenotype (M1) CD163+/CD11b/c+, where the increase of these are observed during cirrhosis and the decreasing during the progression of cirrhosis and markedly with the treatment; suggesting that the increased “increased macrophage” in FIG. 6A are not inflammatories.

FIG. 7 refers to one of the indicators of the type of activated macrophages, the arginase. FIG. 7 shows in a Western blot of arginase that there are more amount of enzyme in the presence of the drug, which is characteristic of macrophage M2s. Therefore, is possible to conclude that adenosine aspartate favors the reduction of inflammatory macrophages M1 and increases expression of arginase favoring, the activation M2.

Using adenosine aspartate, such as demonstrated herein, it has its efficacy in the reversal and prevention of liver fibrosis and of other fibrotic mechanisms associated with inflammation by modulating the state of activation of the macrophage and of the release of the main mediator molecules of the immune response such as cytokines.

1. A method for differential activation of phenotype macrophage M2 in inflammatory fibrogenic processes and its subsequent reversal, in humans, without showing side effects comprising administering adenosine aspartate to the human in need thereof.

2. The method of claim 1, wherein the adenosine aspartate also reduces the phenotype macrophages M1 and achieves conversion to phenotype macrophages M2.

3. The method of claim 1, wherein the adenosine aspartate allows the activation and recruitment of monocytes.

4. The method of claim 1, wherein further the adenosine aspartate increases the levels of IL-10 in humans.

5. A pharmaceutical composition for the treatment of inflammatory-fibrogenic disorders in humans comprising adenosine aspartate.

6. A pharmaceutical composition comprising a therapeutically effective amount of adenosine aspartate in combination with one or more pharmaceutically acceptable excipients.
